(19) United States
(12) Patent Application Publication
(10) Pub. No.: US 2021/0401374 A1
(43) Pub. Date: Dec. 30, 2021

(54) EKG LEAD ATTACHMENT WITH VISUAL AID

(71) Applicant: Christina Redford, Charlotte, NC (US)

(72) Inventor: Christina Redford, Charlotte, NC (US)

(21) Appl. No.: 16/914,368

(22) Filed: Jun. 28, 2020

(51) Int. Cl. A61B 5/00; A61B 5/0428; A61B 50/30; A61B 90/92

(52) U.S. Cl. CPC A61B 5/6841; A61B 5/04286; A61B 2562/222; A61B 90/92; A61B 5/6823; A61B 50/30

(57) ABSTRACT

A electrocardiogram (EKG) lead attachment with a visual aid device including a plurality of retractable leads, a housing member, a plurality of label designations, a plurality of label designation tags, a lead placement visual aid, and a plurality of color tags. The lead placement visual aid includes an anatomical image of a chest cavity including color coordinated label designations which are each positioned on the anatomical image at a location where a corresponding lead in the first set should be placed on the human body. The plurality of color tags are coordinated to match the colors of the color coordinated label designations in the visual aid. The color designations and tags are used to provide an easy to follow visual aid to an operator for placement of the leads on a human body.

### FIELD OF THE INVENTION

Embodiments described herein generally relate to electrocardiogram (EKG) attachment devices, and more particularly to an EKG lead attachment with visual aid.

### BACKGROUND OF THE INVENTION

An electrocardiogram (EKG) machine is an essential tool use in the medical field to examine signs of any heart disease. However, there are several wire leads that are connected to the EKG machine which tends to hang freely. This often causes the hanging leads to tangle together frequently which then requires the medical personnel to untangle each wire lead while constantly checking to ensure they have proper lead. Moreover, there may be emergency situations where an EKG is needed to assess an individual. In those situations, human error may cause an improper reading due to incorrect placement of the electrodes on the individual's body. Hence, an improved way to use the EKG is desirable.

### BRIEF DESCRIPTION OF THE DRAWINGS

The various advantages of the embodiments of the present disclosure will become apparent to one skilled in the art by reading the following specification and appended claims, and by referencing the following drawing(s), in which:

FIG. 1 shows an exemplary view of an EKG lead attachment with visual aid device according to an embodiment of the present disclosure.

FIG. 2 shows another exemplary view of the EKG lead attachment with visual aid device according to an embodiment of the present disclosure.

FIG. 3 shows an exemplary view of a clip in the EKG lead attachment with visual aid device according to an embodiment of the present disclosure.

### SUMMARY OF THE INVENTION

Exemplary embodiments disclosed herein describe an electrocardiogram (EKG) lead attachment with a visual aid device including a plurality of retractable leads, a housing member, a plurality of label designations, a plurality of label designation tags, a lead placement visual aid, and a plurality of color tags. The plurality of leads include a first set of leads which are configured for attachment to a chest cavity region of a human body and a second set of leads which are configured for attachment to a non-chest cavity region of a human body. The housing member encloses a plurality of retractable reels. Each retractable reel having one of the plurality of leads spooled thereon. The housing member includes a plurality of apertures, wherein each aperture corresponds to a stored retractable reel, and the distal end of the spooled lead on the corresponding retractable reel is extended from the aperture so that an operator may access the lead. The plurality of label designations are integrated on a top surface of the housing member, wherein each label designation is positioned above a corresponding one of the plurality of apertures to identify a label identifier for the corresponding lead. The plurality of label designation tags are attached to a distal end of one of the plurality of leads. Each label designation tag corresponds to one of the plurality of label designations integrated on the housing member. The lead placement visual aid is integrated on a top surface of the housing member to aid an operator of the device in placement of the first set of leads, the visual aid including an anatomical image of a chest cavity including color coordinated label designations which are each positioned on the anatomical image at a location where a corresponding lead in the first set should be placed on the human body. The plurality of color tags are coordinated to match the colors of the color coordinated label designations in the visual aid. Each lead in the first set of leads includes one of the plurality of color tags attached at its distal end, and wherein the attached color tag for the lead matches the color of the color coordinated label designation which corresponds to the label designation tag for the lead.

In some exemplary embodiments, each lead is extendible from the housing member.

In some exemplary embodiments, each extended lead is retractable into the housing member.

In some exemplary embodiments, the color coordinated label designations each correspond to one of the plurality of label designations integrated on the housing member.

In some exemplary embodiments, each lead has a metal rod installed at the distal end of the lead.

In some exemplary embodiments, each lead has a stopper attached at its distal end above the metal rod.

In some exemplary embodiments, the device includes a plurality of clips which are each attached to the metal rod of a lead.

In some exemplary embodiments, the housing member includes a pair of clip holes for attaching the device to a EKG machine.

In some exemplary embodiments, the housing member includes a plug which attached to a mating plug on a EKG machine for transferring data and signals between the EKG machine and the device.

In some exemplary embodiments, the plurality of label designations identify a non-chest cavity region of a human body to where a corresponding lead should be attached.

In some exemplar embodiments, the plurality of label designations identify a non-chest cavity region include “RA”, “RL”, “LA” and “LL”.

### DETAILED DESCRIPTION

The present disclosure relates to a portable electrocardiogram (“EKG”) lead attachment with visual aid device (“the device”). The main purpose of the device is to reduce the amount of time it takes to perform an EKG reading and with minimal errors. The device includes retractable leads to prevent tangled leads which delays the time it takes to perform an EKG reading. Moreover, the device includes a visual aid for quick and easy to follow guidance for placement of the leads. The visual aid includes color coordination so that an operator of the device may quickly ascertain where a lead should be placed.

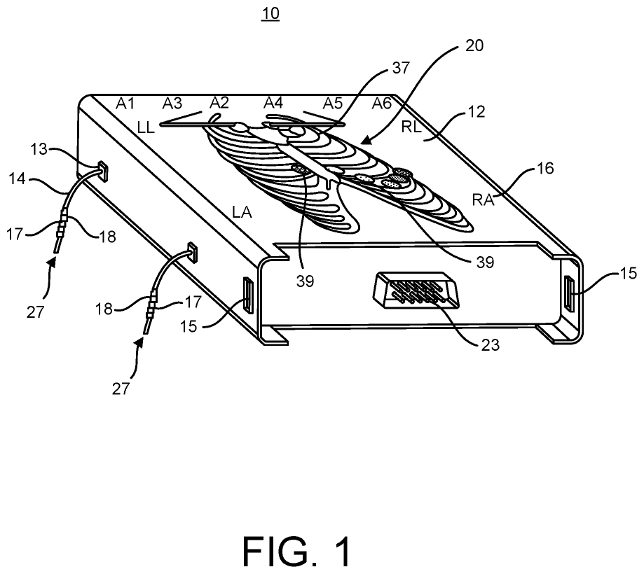

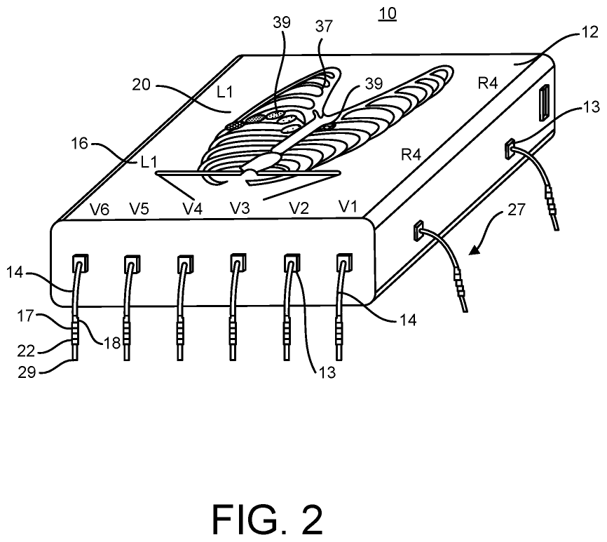

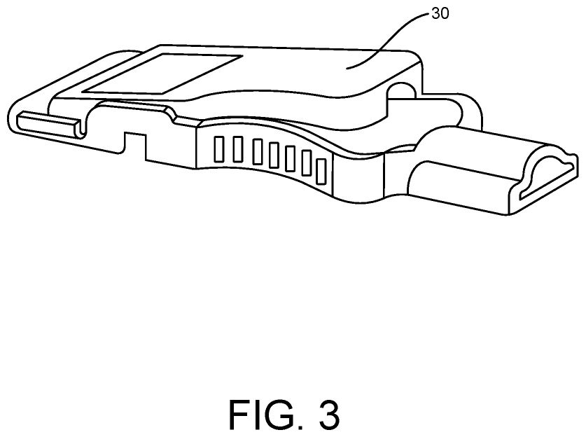

As illustrated in FIGS. 1-3, the device 10 includes a housing member 12, a plurality of leads 14, a plurality of label designations 16, a plurality of label designation tags 17, a plurality of stoppers 18, a lead placement visual aid 20, and a plurality of color tags 22. The housing member 12 encloses a plurality of retractable reels, a plurality of apertures 13, a pair of clip holes 15, and a plug 23. The housing member may be made in any suitable size. Moreover, the housing member may be made of any suitable material, such as, for example, plastic.

Each retractable reel has one of the plurality of leads 14 spooled thereon. The spooled lead may be extended from the reel and the extended lead may be retracted to the reel. The retractable reel may include a spool for storing the reeled lead. The main mechanical function of the retractable reel may be performed by a ratcheting mechanism that stops the rotation of the reel and by a condensed spring coil that applies a torsional force to retract the lead 14 back into the casing for the reel.

The plurality of apertures 13 each correspond to one of the retractable reels enclosed in the housing member 12. The distal end 27 of a spooled lead on a corresponding retractable reel is extended from the corresponding aperture 13 so that an operator of the device may access the lead 14 for placement for an EKG reading. The apertures 13 are located along the sides of the housing member 12 to prevent any complications and/or tangling with lead placement.

The pair of clip holes 15 may be located at opposite ends along one side of the housing unit for attaching the device to a EKG machine. Moreover, the housing member 12 includes a plug 23, such as, for example, a male VGA plug. The plug attaches to a mating plug on a EKG machine for transferring data and signals between the EKG machine and the device.

The plurality of leads 14 each have a stopper 18 attached to its distal end above the metal rod 29. The stopper blocks the lead from slamming up against the EKG device when the lead 14 is retracted. Moreover, the plurality of leads 14 each include a metal rod 29 installed at the distal end 27 of the lead for attachment to one of the clips 30. The plurality of clips 30 attach to the electrodes that are attached to patient. Each lead 14 is attached to an electrode placed on a patient's body at a particular place via clip 30. The plurality of leads include a first set of leads which are configured for attachment to a chest cavity region of a human body and a second set of leads which are configured for attachment to a non-chest cavity region of a human body, such as, for example, an arm or a leg.

The housing member 12 includes a plurality of label designations 16 integrated on a top surface of the housing member. Each label designation is positioned above a corresponding one of the plurality of apertures 13 to identify a label identifier for the corresponding lead 14. So, for example, a label designation “V1” may be integrated on the housing member above the aperture 13 corresponding to a lead 14 which will be identified as label “V1”. Some of the label destinations 16 actually identify a non-chest cavity region of a human body to where a corresponding lead should be attached, such as, for example, “RA” which corresponds to right arm, “RL” which corresponds to right leg, “LA” which corresponds to left arm, and “LL” which corresponds to left leg.

The device 10 includes a plurality of label designation tags 17 which are each attached to a distal end 27 of one of the plurality of leads 14. Each label designation tag 17 corresponds to one of the plurality of label designations integrated on the housing member 12.

The housing member 12 includes a lead placement visual aid 20 integrated on its top surface. The lead placement visual aid provides guidance to an operator of the device 10 in placement of the first set of leads on human body (i.e., patient). The visual aid includes an anatomical image of a chest cavity 37 including color coordinated label designations 39 which are each positioned on the anatomical image at a location where a corresponding lead 14 in the first set should be placed on the human body. The color coordinated label designations each correspond to one of the plurality of label designations 16 integrated on the housing member

The first set of leads each include a color tag 22 attached at its distal end 27. The attached color tag 22 on a particular lead 14 in the first set matches the color of a corresponding color coordinated label designation 39 in the lead placement visual aid 20 and the corresponding color coordinated label designation 39 is the color coordinated label designation which corresponds to the label designation 16 for the lead. The plurality of color tags 22 are each color coordinated to match the color of a corresponding color coordinated label designations in the visual aid 20.

The disclosed embodiments are described for a portable device which is attached to an EKG machine and which may be detached from the EKG machine. In an alternative embodiment, the device 10 is fixedly integrated into the EKG machine.

Moreover, it should be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. An electrocardiogram (EKG) lead attachment with a visual aid device comprising:
a plurality of leads, including a first set of leads which are configured for attachment to a chest cavity region of a human body and a second set of leads which are configured for attachment to a non-chest cavity region of a human body;
a housing member for enclosing a plurality of retractable reels, each retractable reel having one of the plurality of leads spooled thereon, the housing member including a plurality of apertures, wherein each aperture corresponds to one of the enclosed retractable reel, and the distal end of the spooled lead on the corresponding retractable reel is extended from the aperture so that an operator may access the lead;
a plurality of label designations integrated on a top surface of the housing member, wherein each label designation is positioned above a corresponding one of the plurality of apertures to identify a label identifier for the corresponding lead;
a plurality of label designation tags which are each attached to a distal end of one of the plurality of leads, wherein each label designation tag corresponds to one of the plurality of label designations integrated on the housing member;
a lead placement visual aid integrated on a top surface of the housing member to aid an operator of the device in placement of the first set of leads, the visual aid including an anatomical image of a chest cavity including color coordinated label designations which are each positioned on the anatomical image at a location where a corresponding lead in the first set should be placed on the human body; and
a plurality of color tags coordinated to match the colors of the color coordinated label designations in the visual aid, wherein each lead in the first set of leads includes one of the plurality of color tags attached at its distal end, and wherein the attached color tag for the lead matches the color of the color coordinated label designation which corresponds to the label designation tag for the lead.

2. The device of claim 1, wherein each lead is extendible from the housing member.

3. The device of claim 1, wherein each extended lead is retractable into the housing member.

4. The device of claim 1, wherein the color coordinated label designations each correspond to one of the plurality of label designations integrated on the housing member.

5. The device of claim 1, wherein each lead has a metal rod installed at the distal end of the lead.

6. The device of claim 5, wherein each lead has a stopper attached at its distal end above the metal rod.

7. The device of claim 5, further comprising a plurality of clips which are each attached to the metal rod of a lead.

8. The device of claim 1, wherein the housing member includes a pair of clip holes for attaching the device to a EKG machine.

9. The device of claim 1, wherein the housing member includes a plug which attaches to a mating plug on a EKG machine for transferring data and signals between the EKG machine and the device.

10. The device of claim 1, wherein some of the plurality of label designations identify a non-chest cavity region of a human body to where a corresponding lead should be attached.

11. The device of claim 8, wherein the plurality of label designations identifying a non-chest cavity region include “RA”, “RL”, “LA” and “LL”.
